SEQUENCE LISTING (6 sequences)

SEQ ID NO: 1 - protein, 277 aa, Staphylococcus aureus
GATEATNATNNQSTQVSQATSQPINFQVQKDGSSEKSHMDDYMQHPGKVIKQNNKYYFQTVLNNASFWKEYKFYNANNQELATTVVNDNKKADTRTINVAVEPGYKSLTTKVHIVVPQINYNHRYTTHLEFEKAIPTLADAAKPNNVKPVQPKPAQPKTPTEQTKPVQPKVEKVKPTVTTTSKVEDNHSTKVVSTDTTKDQTKTQTAHTVKTAQTAQEQNKVQTPVKDVATAKSESNNQAVSDNKSQQTNKVTKHNETPKQAGPSKDSKAKELPKTG

SEQ ID NO: 2 - protein, 570 aa, Staphylococcus aureus
MSNGEAQAAAEETGGTNTEAQPKTEAVASPTTTSEKAPETKPVANAVSVSNKEVEAPTSETKEAKEVKEVKAPKETKEVKPAAKATNNTYPILNQELREAIKNPAIKDKDHSAPNSRPIDFEMKKKDGTQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTEEDYKAEKLLAPYKKAKTLERQVYELNKIQDKLPEKLKAEYKKKLEDTKKALDEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPTKGEVESSSTTPTKVVSTTQNVAKPTTASSKTTKDVVQTSAGSSEAKDSAPLQKANIKNTNDGHTQSQNNKNT

SEQ ID NO: 3 - protein, 121 aa, Staphylococcus aureus
FTFEPFPTNEEIESNKKMLEKEKAYKESFKNSGLPTTLGKLDERLRNYLKKGTKNSAQFEKMVILTENKGYYTVYLNTPLAEDRKNVELLGKMYKTYFFKKGESKSSYVINGPGKTNEYAY

SEQ ID NO: 4 - protein, 84 aa, Staphylococcus aureus
STSLPTSNEYQNEKLANELKSLLDELNVNELATGSLNTYYKRTIKISGQKAMYALKSKDFKKMSEAKYQLQKIYNEIDEALKSY

SEQ ID NO: 5 - protein, 7 aa, Artificial Sequence
  Synthetic construct
MHHHHHH

SEQ ID NO: 6 - protein, 14 aa, Artificial Sequence
  Synthetic construct
LNDIFEAQKIEWHG

(19) United States
(12) Patent Application Publication
(10) Pub. No.: US 2018/0143194 A1
(43) Pub. Date: May 24, 2018

(54) COMPOSITIONS AND METHODS FOR DETECTING STAPHYLOCOCCOUS INFECTIONS

(71) Applicants: THE UNIVERSITY OF ROCHESTER, Rochester, NY (US); EMORY UNIVERSITY, Atlanta, GA (US)

(72) Inventors: John Linforth DAISS, Rochester, NY (US); Edward M. SCHWARZ, Rochester, NY (US); Kohei NISHITANI, Yamashina Ku, Kyoto (JP); Sandeep SOIN, Rochester, NY (US); Frances Eun-Hyung LEE, Atlanta, GA (US)

(21) Appl. No.: 15/563,667

(22) PCT Filed: Apr. 4, 2016
(86) PCT No.: PCT/US2016/025885

§ 371 Date: Oct. 2, 2017

Related U.S. Application Data
(60) Provisional application No. 62/142,059, filed on Apr. 2, 2015.

(51) Int. Cl. G01N 33/569; G01N 33/50; G01N 33/536; C07K 16/12

(52) U.S. Cl. CPC G01N 33/56938; G01N 33/5091; C07K 2317/14; C07K 16/1271; G01N 33/536

(57) ABSTRACT

Provided herein are compositions and methods for detecting and/or diagnosing a Staphylococcus aureus (S. aureus) infection in a subject. It is a surprising finding of the present invention that an S. aureus infection may be detected by using one or more antigens selected from the group consisting of an IsdA antigen, IsdB antigen, CHIPS antigen, and a SCIN antigen. In some embodiments, these S. aureus antigens are used to detect antibodies secreted by the plasmablasts from the subject.

### STATEMENT REGARDING FEDERALLY SPONSORED RESEARCH OR DEVELOPMENT

This invention was made with Government support under Contract No. P30AR061307 awarded by the National Institutes of Health. The U.S. Government has certain rights in this invention.

### BACKGROUND OF THE INVENTION

### 1) Field of the Invention

The field of the invention is detecting infection in a subject.

### 2) Description of Related Art

Infected total joint replacements (TJR) are a significant source of cost and morbidity. Facing years of limited and declining mobility, millions of Americans have had their lives transformed by TJR. In fact, the success of these surgeries has been heralded as the greatest medical achievement of the second half of the Twentieth Century. In 2014, as many as 1.5 million Americans will undergo replacement of a knee or hip and these numbers will grow for the foreseeable future as the population ages and lives longer [Cram P, Lu X, Kates S L, et al., JAMA. 2012 Sep. 26, 308(12):1227-1236; Kurtz S M, Lau E, Ong K, et al., Clin Orthop Relat Res. 2009, October, 467(10):2606-2612]. However, the introduction of a large foreign body into these patients creates an ideal niche for opportunistic pathogens, so it is not surprising that infection is by far the most feared complication of TJR [Berbari E F, Osmon D R, Lahr B, et al., Infect. Control Hosp. Epidemiol. 2012 August, 33(8):774-781]. Following years of surgical vigilance, the rate of TJR infection has been reduced to 1-2% for total hip and total knee replacements [Ong K L, Kurtz S M, Lau E, et al. J Arthroplasty. 2009 Sep. 24(6 Suppl):105-109; Parvizi J, Azzam K, Ghanem E, et al. Clin Orthop Relat Res. 2009 July, 467(7):1732-1739; Parvizi J, Della Valle C J. AAOS Clinical, J Am Acad Orthop Surg. 2010 December, 18(12):771-772]. Even with such a modest rate of infection, 15,000 to 30,000 Americans will have their dreams of improved independence and mobility shattered and the healthcare system will absorb billions of dollars in additional costs [Anderson D J, Kaye K S, Chen L F, et al. PLoS One. 2009, 4(12):e8305]. For many patients with infected TJR the outcomes can be severe: between 20 and 40% will end in fusion, amputation or death from infection [Parvizi J, Azzam K, Ghanem E, et al. Clin Orthop Relat Res. 2009 July, 467(7):1732-1739; Cahill J L, Shadbolt B, Scarvell J M, et al. J Orthop Surg (Hong Kong). 2008 April, 16(1):58-65].

Numerous bacterial species cause TJR infections, but as many as 80% involve Staphylococci in single or mixed infections, and these are about evenly divided between coagulase-positive and coagulase-negative species (Moran E, Byren I, Atkins B L. J Antimicrob Chemother. 2010 November, 65 Suppl 3:iii45-54). Most non-Staphylococci and the coagulase negative Staphylococci respond well to antibiotic therapy. Conversely, in the context of TJR, S. aureus (and especially methicillin resistant S. aureus; MRSA) is recalcitrant to antibiotics and accounts for the most challenging and costly clinical cases [Parvizi J, Pawasarat I M, Azzam K A, et al. J Arthroplasty. 2010 September, 25(6 Suppl):103-107; Salgado C D, Dash S, Cantey J R, et al. Clin Orthop Relat Res. 2007 August, 461:48-53; Teterycz D, Ferry T, Lew D, et al. Int J Infect Dis. 2010 October, 14(10):e913-918].

Diagnosis of infected TJR is complicated by several factors [Moran E, Byren I, Atkins B L. J Antimicrob Chemother. 2010 November, 65 Suppl 3:iii45-54. Della Valle C, Parvizi J, Bauer T W, et al. Diagnosis of periprosthetic joint infections of the hip and knee. J Am Acad Orthop Surg. 2010 December; 18(12):760-770]. First, it is uncommon; only one or two patients in one hundred will develop infections. Second, early infections look similar to normal healing with elevated inflammatory markers, redness, pain and swelling at the wound site. For several weeks most infections are not distinguishable from normal healing. Third, the clinical onset is often delayed for months, sometimes years. Fourth, it often presents similarly to other common conditions including seroma, hematoma, deep venous thrombosis or superficial surgical site infection such as cellulitis. Rarely do these infections present in a fulminant manner so providers must wait for the infection to declare itself. Thus, diagnosis typically occurs after the acute phase has passed and a chronic infection is already well established.

Critically, it is often difficult [DeBonville D. MLO Med Lab Obs. 2012 January, 44(1):26-27] and expensive to obtain a pathogen-containing sample that can be easily tested by culture or PCR and the clinician is compelled to use indirect measures. In fact, the Musculoskeletal Infection Society recently formed a workgroup on diagnostic criteria [Parvizi J, Zmistowski B, Berbari E F, et al. Clin Orthop Relat Res. 2011 November, 469(10:2992-2994]. They proposed the following criteria: sinus tract drainage, two separate positive cultures from the joint, or when four of the following six conditions are met: 1) increased serum erythrocyte sedimentation rate (ESR) and C-reactive protein (CRP); 2) increased synovial leukocyte count; 3) elevated neutrophil percentage; 4) purulence in the joint; 5) isolation of an organism in one culture of joint fluid of tissue; 6) greater than five neutrophils per high-power field of histologic analysis of synovial tissue. While these guidelines are designed to help the physician make accurate treatment decisions, the criteria are non-specific and are limited by the current clinical tests available.

Early and precise diagnosis of infection is essential. Precise diagnosis obviates the need for empiric broad-spectrum therapies, which have the potential to lead to future pathogen resistance. Furthermore, minimizing the need for empiric broad-spectrum antibiotic therapy with precise diagnosis limits the side effects of unneeded antibiotic therapy such as Clostridium difficile infection or renal injury due to antibiotic toxicities [Gonzalez de Molina F J, Ferrer R. Crit Care. 2011, 15(4):175; Yarinsky S, Wheeler W E. W V Med J. 1990 June, 86(6):239-242]. As the pharmaceutical industry continues to make advances in pathogen-specific therapies, the field of diagnostics needs to keep up.

### SUMMARY OF THE INVENTION

Provided herein is a method of determining an S. aureus infection in a subject, comprising: 1) generating a plasmablasts-secreted antibody medium by collecting peripheral blood mononuclear cells (PBMCs) from blood in a subject, removing existing antibodies from the PBMCs and culturing the antibody-free PBMCs in a medium for a period of time to allow secretion of antibodies by plasmablasts contained in the PBMCs into the medium; 2) contacting the plasmablasts-secreted antibody medium with one or more S. aureus antigens; 3) detecting the presence of one or more antigen-specific antibodies in the plasmablasts-secreted antibody medium at a level above a control; and 4) determining the S. aureus infection in the subject based on the detection of antigen-specific antibodies in step 3). In some embodiments, the one or more S. aureus antigens are selected from an IsdA antigen, IsdB antigen, CHIPS antigen, and SCIN antigen. The plasmablasts-secreted antibody medium can be contacted with one, or two, or three, or all of the IsdA antigen, IsdB antigen, CHIPS antigen, and SCIN antigen.

The plasmablasts-secreted antibody medium can be generated by culturing antibody-free PBMCs for about 1 to about 168 hours. In some embodiments, the antibody-free PBMCs are cultured for about 36 to about 72 hours. In some embodiments, one or more antigen-specific antibodies in the plasmablasts-secreted antibody medium are at a level of at least about 10% more than an antibody control level.

Also provided herein is a kit for determining if a subject has a Staphylococcus aureus (S. aureus) infection, wherein the kit includes one or more antigens selected from the group consisting of an IsdA antigen, an IsdB antigen, a CHIPS antigen, and a SCIN antigen; a reservoir for contacting the one or more antigens with the plasmablasts-secreted antibody medium; and one or more compositions for detecting binding between the one or more antigens with one or more antibodies in the plasmablasts-secreted antibody medium. In some embodiments, all of the IsdA antigen, IsdB antigen, CHIPS antigen, and SCIN antigen are comprised within the kit. In some embodiments, the composition for detecting binding is a labelled anti-IgG antibody.

### BRIEF DESCRIPTION OF THE DRAWINGS

FIGS. 1A and 1B show that Human IgG levels against IsdB are the best single antigen predictor of recent/ongoing infection and they are not clinically useful. A) Dot plot presenting anti-IsdB IgG titers for 40 Controls and 35 patients on a log 10 scale. B) ROC curve presenting the same data. AUC=0.80.

FIG. 2 shows that the multiplex immunoassay described herein accurately identifies patients with ongoing S. aureus infections. FI values for 14 antigens in 16 patients, twelve with suspected S. aureus infections and four non-infected controls. Patients 1-4 are non-infected controls; 5-12 were culture-confirmed with S. aureus; 13 culture-confirmed with P. aeruginosa; 14 and 15, >30 days antibiotic therapy; 16, culture negative. Red line=positive-negative cut-off=twice highest FI value measured for any antigen in a control sample.

FIG. 3 shows the S. aureus antigens that were detected by IgG in the MENSA of infected patients. FI values that were at least twice the maximum FI value for any antigen among the non-infected controls are presented as filled-in red boxes.

### DETAILED DESCRIPTION OF THE INVENTION

Provided herein are compositions and methods for detecting and/or diagnosing a Staphylococcus aureus (S. aureus) infection in a subject. It is a surprising finding of the present invention that an S. aureus infection may be detected by using one or more antigens selected from the group consisting of an IsdA antigen, IsdB antigen, CHIPS antigen, and a SCIN antigen. In some embodiments, these S. aureus antigens are used to detect antibodies secreted by the plasmablasts from the subject.

### Definitions

Terms used throughout this application are to be construed with ordinary and typical meaning to those of ordinary skill in the art. However, Applicants desire that the following terms be given the particular definition as defined below.

As used in the specification and claims, the singular form “a,” “an,” and “the” include plural references unless the context clearly dictates otherwise. For example, the term “a cell” includes a plurality of cells, including mixtures thereof.

The terms “about” and “approximately” are defined as being “close to” as understood by one of ordinary skill in the art. In one non-limiting embodiment the terms are defined to be within 10%. In another non-limiting embodiment, the terms are defined to be within 5%. In still another non-limiting embodiment, the terms are defined to be within 1%.

The term “antibody” is used in the broadest sense, and specifically covers monoclonal antibodies (including full length monoclonal antibodies), polyclonal antibodies, and multispecific antibodies (e.g., bispecific antibodies). Antibodies (Abs) and immunoglobulins (Igs) are glycoproteins having the same structural characteristics. While antibodies exhibit binding specificity to a specific target, immunoglobulins include both antibodies and other antibody-like molecules which lack target specificity. Native antibodies and immunoglobulins are usually heterotetrameric glycoproteins of about 150,000 daltons, composed of two identical light (L) chains and two identical heavy (H) chains. Each heavy chain has at one end a variable domain (V_{H}) followed by a number of constant domains. Each light chain has a variable domain at one end (V_{L}) and a constant domain at its other end.

The term “antibody fragment” refers to a portion of a full-length antibody, generally the target binding or variable region. Examples of antibody fragments include Fab, Fab′, F(ab′)₂ and Fv fragments. The phrase “functional fragment or analog” of an antibody is a compound having qualitative biological activity in common with a full-length antibody. For example, a functional fragment or analog of an anti-IgE antibody is one which can bind to an IgE immunoglobulin in such a manner so as to prevent or substantially reduce the ability of such molecule from having the ability to bind to the high affinity receptor, FcεRI. As used herein, “functional fragment” with respect to antibodies, refers to Fv, F(ab) and F(ab′)₂ fragments. An “Fv” fragment is the minimum antibody fragment which contains a complete target recognition and binding site. This region consists of a dimer of one heavy and one light chain variable domain in a tight, non-covalent association (V_{H}-V_{L} dimer). It is in this configuration that the three CDRs of each variable domain interact to define an target binding site on the surface of the V_{H}-V_{L} dimer. Collectively, the six CDRs confer target binding specificity to the antibody. However, even a single variable domain (or half of an Fv comprising only three CDRs specific for a target) has the ability to recognize and bind target, although at a lower affinity than the entire binding site. “Single-chain Fv” or “sFv” antibody fragments comprise the V_{H} and V_{L} domains of an antibody, wherein these domains are present in a single polypeptide chain. Generally, the Fv polypeptide further comprises a polypeptide linker between the V_{H} and V_{L} domains which enables the sFv to form the desired structure for target binding.

The Fab fragment contains the constant domain of the light chain and the first constant domain (CH1) of the heavy chain. Fab′ fragments differ from Fab fragments by the addition of a few residues at the carboxyl terminus of the heavy chain CH1 domain including one or more cysteines from the antibody hinge region. F(ab′) fragments are produced by cleavage of the disulfide bond at the hinge cysteines of the F(ab′)₂ pepsin digestion product. Additional chemical couplings of antibody fragments are known to those of ordinary skill in the art.

The term “B cell” includes B-1 cells, B-2 cells, plasma B cells, memory B cells, and plasmablasts. The term “peripheral blood B cell” refers to a B cell present in the peripheral blood of an individual. Peripheral blood B cells include, but are not limited to, immature/transitional B cells, naïve B cells, memory B cells, and plasmablasts.

The terms “cell,” “cell line” and “cell culture” include progeny. It is also understood that all progeny may not be precisely identical in DNA content, due to deliberate or inadvertent mutations. Variant progeny that have the same function or biological property, as screened for in the originally transformed cell, are included.

A “composition” is intended to include a compound, an active agent, or a combination of active agent with another agent or compound or composition, inert (for example, a detectable agent or label) or active, such as an adjuvant.

As used herein, the term “comprising” is intended to mean that the compositions and methods include the recited elements, but not excluding others. “Consisting essentially of” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

A “control” is an alternative subject or sample used in an experiment for comparison purpose. A control can be “positive” or “negative.”

As used herein, the term “culture supernatant” refers to a medium in which a cell or cells have developed or proliferated. In some embodiments, the culture supernatant is a plasmablasts-secreted antibody medium obtained following approximately 30 minutes, 1 hour, 2 hours, 4 hours, 8 hours, 12 hours, 24 hours, 36 hours, 48 hours, 72 hours, 84 hours, 108 hours, 132 hours, 156 hours, or 168 hours of cell development or proliferation in the medium.

The word “label” when used herein refers to a detectable compound or composition which can be conjugated directly or indirectly to a molecule or protein, e.g., an antibody. The label may itself be detectable (e.g., radioisotope labels or fluorescent labels) or, in the case of an enzymatic label, may catalyze chemical alteration of a substrate compound or composition which is detectable.

The term “lymphocyte” refers to natural killer cells, T cells, or B cells.

“Mammal” for purposes of treatment refers to any animal classified as a mammal, including human, domestic and farm animals, nonhuman primates, and zoo, sports, or pet animals, such as dogs, horses, cats, cows, etc.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single target site. Furthermore, in contrast to conventional (polyclonal) antibody preparations which typically include different antibodies directed against different determinants (epitopes), each monoclonal antibody is directed against a single determinant on the target. In addition to their specificity, monoclonal antibodies are advantageous in that they may be synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies for use with the present invention may be isolated from phage antibody libraries using the well-known techniques. The parent monoclonal antibodies to be used in accordance with the present invention may be made by the hybridoma method first described by Kohler and Milstein, Nature 256, 495 (1975), or may be made by recombinant methods.

The terms “peripheral blood mononuclear cell” and “PBMC” are used interchangeably herein and refer to as any blood cell with a round nucleus (i.e. a lymphocyte, a monocyte, or a macrophage). As used herein, the term “PBMC culture” refers to any PBMC obtained from a subject's blood that grow or develop in a medium in vitro. A “PBMC culture supernatant” refers herein to a medium in which the existing antibodies in the PBMC-containing blood is removed and obtained from a subject's blood have developed or proliferated.

“Plasmablasts” are defined herein as white blood cells that secrete large volumes of antibodies. They are antibody-secreting B cells that may be identified by flow cytometry as CD19^{low}CD20⁻CD27^{high}CD38^{high}CD138^{±}. In some embodiments, plasmablasts appear in the blood shortly after infection and persist as long as antigen is detectable. In some embodiments, plasmablasts have differentiated from memory B cells.

The term “polypeptide” is used in its broadest sense to refer to a compound of two or more subunit amino acids, amino acid analogs, or peptidomimetics. The subunits may be linked by peptide bonds. In another embodiment, the subunit may be linked by other bonds, e.g. ester, ether, etc. As used herein the term “amino acid” refers to either natural and/or unnatural or synthetic amino acids, including glycine and both the D or L optical isomers, and amino acid analogs and peptidomimetics. A peptide of three or more amino acids is commonly called an oligopeptide if the peptide chain is short. If the peptide chain is long, the peptide is commonly called a polypeptide or a protein.

As used herein, the term “solid support” means a non-aqueous matrix to which the one or more S. aureus antigens of the present invention can adhere and is not limited to a specific type of support. Rather a large number of supports are available and are known to one of ordinary skill in the art. Solid phase supports include silica gels, resins, derivatized plastic films, glass beads, cotton, plastic beads, and alumina gels. As used herein, “solid support” also includes synthetic antigen-presenting matrices, cells, and liposomes. In certain embodiments, depending on the context, the solid phase can comprise the well of an assay plate; in others it is a purification column (e.g. an affinity chromatography column). A suitable solid support may be selected on the basis of desired end use and suitability for various protocols.

The term “subject” is defined herein to include animals such as mammals, including, but not limited to, primates (e.g., humans), cows, sheep, goats, horses, dogs, cats, rabbits, rats, mice and the like. In some embodiments, the subject is a human.

The term “variable” in the context of variable domain of antibodies, refers to the fact that certain portions of the variable domains differ extensively in sequence among antibodies and are used in the binding and specificity of each particular antibody for its particular target. However, the variability is not evenly distributed through the variable domains of antibodies. It is concentrated in three segments called complementarity determining regions (CDRs) also known as hypervariable regions both in the light chain and the heavy chain variable domains. The more highly conserved portions of variable domains are called the framework (FR). The variable domains of native heavy and light chains each comprise four FR regions, largely a adopting a .beta.-sheet configuration, connected by three CDRs, which form loops connecting, and in some cases forming part of, the .beta.-sheet structure. The CDRs in each chain are held together in close proximity by the FR regions and, with the CDRs from the other chain, contribute to the formation of the target binding site of antibodies (see Kabat et al.) As used herein, numbering of immunoglobulin amino acid residues is done according to the immunoglobulin amino acid residue numbering system of Kabat et al., (Sequences of Proteins of Immunological Interest, National Institute of Health, Bethesda, Md. 1987), unless otherwise indicated.

### DETAILED DESCRIPTION

As indicated above, provided herein are compositions and methods for detecting and/or diagnosing a Staphylococcus aureus (S. aureus) infection in a subject. It is a surprising finding of the present invention that an S. aureus infection may be detected by using an S. aureus antigen and a peripheral blood mononuclear cell (PBMC) culture supernatant wherein the PBMC is obtained from the subject. In some embodiments, one or more S. aureus antigens is selected from the group consisting of an IsdA antigen, an IsdB antigen, a CHIPS antigen, and a SCIN antigen.

Provided herein is a method of determining S. aureus infection in a subject, comprising: 1) generating a plasmablasts-secreted antibody medium by collecting peripheral blood mononuclear cells (PBMCs) from blood in a subject, removing existing antibodies from the PBMCs and culturing the antibody-free PBMCs in a medium for a period of time to allow secretion of antibodies by plasmablasts contained in the PBMCs into the medium; 2) contacting the plasmablasts-secreted antibody medium with one or more S. aureus antigens; 3) detecting the presence of one or more antigen-specific antibodies in the plasmablasts-secreted antibody medium at a level above a control; and 4) determining the S. aureus infection in the subject based on the detection of antigen-specific antibodies in step 3).

In some embodiments, the PBMCs are isolated from a subject's blood sample via a Ficoll-Paque method. Ficoll-Paque methods of PBMC isolation are well known to those of ordinary skill in the art, and all such methods are included herein. In some embodiments, the Ficoll-Pacque method includes the use of POLYMORPHPREP™. In some embodiments, the antibody-free PBMCs are cultured in a medium at approximately 37° C. for approximately 12 hours, 24 hours, 36 hours, 48 hours, 72 hours, or 84 hours. In some embodiments, the medium is a DMEM, RPMI 1640, and SFM₄MAB (Hyclone Laboratories, Serum Free Medium for Monoclonal Antibodies). In some embodiments, the medium is supplemented with a fetal bovine serum. The fetal bovine serum may be at about 5% v/v, about 10% v/v, about 15% v/v, about 20% v/v, or about 25% v/v. Antibiotics such as gentamicin may also be added to the medium. In some embodiments, the PBMCs are cultured in the presence of about 5% CO₂.

In some embodiments, the plasmablasts is optionally further removed from the plasmablasts-secreted antibody medium to generate a substantially cell-free medium.

As used herein, “a control level” refers to a level of antigen-antibody binding in the plasmablasts-secreted antibody medium from a healthy, non-infection subject or a subject with an infection that has been cleaned. As used herein, the term “at a level above a control level” refers to a level of antigen-antibody binding that is at least about 10% more, about 15% more, about 20% more, about 30% more, about 40% more, about 50% more, about 60% more, about 70% more, about 80% more, about 90% more, or about 100% more than the control level. In one embodiment, the term “at a level above a control level” means a level of greater than 10%, 20%, 30%, 40%, 50%, 60% 70%, 80%, 90%, 100%, or 150% or 200% more than the control level.

In some embodiments, the S. aureus antigens are selected from the group consisting of an IsdA antigen, an IsdB antigen, a CHIPS antigen, and a SCIN antigen. In some embodiments, the plasmablasts-secreted antibody medium is contacted with two or three or all of an IsdA antigen, an IsdB antigen, a CHIPS antigen, and a SCIN antigen.

The term “IsdA” refers to an S. aureus surface or cell wall expressed polypeptide also referred to as iron-regulated surface determinant protein A. In some embodiments, the IsdA antigen comprises the sequence of SEQ ID NO:1, or a polypeptide sequence having at or greater than about 80%, about 85%, about 90%, about 95%, or about 98% homology with SEQ ID NO:1, or a polypeptide comprising a portion of SEQ ID NO:1. In some embodiments, the IsdA antigen comprises additional polypeptide sequences that allow for purification of the antigen and/or attachment of the antigen to a solid support. In some embodiments, the IsdA antigen comprises a polypeptide sequence of SEQ ID NO:1 and an N-terminal six histidine purification sequence of MHHHHHH (SEQ ID NO:5). In other embodiments, the IsdA antigen comprises a polypeptide sequence of SEQ ID NO:1 and a C-terminal biotinylation sequence of LNDIFEAQKIEWHG (SEQ ID NO:6). In some embodiments, the IsdA antigen comprises SEQ ID NO:1, SEQ ID NO: 5 and SEQ ID NO:6.

The term “IsdB” refers to an S. aureus surface or cell wall expressed polypeptide also referred to as iron-regulated surface determinant protein B. In some embodiments, the IsdB antigen comprises the sequence of SEQ ID NO:2, or a polypeptide sequence having at or greater than about 80%, about 85%, about 90%, about 95%, or about 98% homology with SEQ ID NO:2, or a polypeptide comprising a portion of SEQ ID NO:2. In some embodiments, the IsdB antigen comprises additional polypeptide sequences that allow for purification of the antigen and/or attachment of the antigen to a solid support. In some embodiments, the IsdB antigen comprises a polypeptide sequence of SEQ ID NO:2 and an N-terminal six histidine purification sequence of MHHHHHH (SEQ ID NO:5). In other embodiments, the IsdB antigen comprises a polypeptide sequence of SEQ ID NO:2 and a C-terminal biotinylation sequence of LNDIFEAQKIEWHG (SEQ ID NO:6). In some embodiments, the IsdB antigen comprises SEQ ID NO:2, SEQ ID NO:5 and SEQ ID NO:6.

The term “CHIPS” refers to an S. aureus exoprotein also referred to as chemotaxis inhibitory protein. In some embodiments, the CHIPS antigen comprises the sequence of SEQ ID NO:3, or a polypeptide sequence having at or greater than about 80%, about 85%, about 90%, about 95%, or about 98% homology with SEQ ID NO:3, or a polypeptide comprising a portion of SEQ ID NO:3. In some embodiments, the CHIPS antigen comprises additional polypeptide sequences that allow for purification of the antigen and/or attachment of the antigen to a solid support. In some embodiments, the CHIPS antigen comprises a polypeptide sequence of SEQ ID NO:3 and an N-terminal six histidine purification sequence of MHHHHHH (SEQ ID NO:5). In other or further embodiments, the CHIPS antigen comprises a polypeptide sequence of SEQ ID NO:3 and a C-terminal biotinylation sequence of LNDIFEAQKIEWHG (SEQ ID NO:6). In some embodiments, the CHIPS antigen comprises SEQ ID NO:3, SEQ ID NO: 5 and SEQ ID NO:6.

The term “SCIN” refers to an S. aureus exoprotein also referred to as staphylococcal complement inhibitor. In some embodiments, the SCIN antigen comprises the sequence of SEQ ID NO:4, or a polypeptide sequence having at or greater than about 80%, about 85%, about 90%, about 95%, or about 98% homology with SEQ ID NO:4, or a polypeptide comprising a portion of SEQ ID NO:4. In some embodiments, the SCIN antigen comprises additional polypeptide sequences that allow for purification of the antigen and/or attachment of the antigen to a solid support. In some embodiments, the SCIN antigen comprises a polypeptide sequence of SEQ ID NO:4 and an N-terminal six histidine purification sequence of MHHHHHH (SEQ ID NO:5). In other or further embodiments, the SCIN antigen comprises a polypeptide sequence of SEQ ID NO:4 and a C-terminal biotinylation sequence of LNDIFEAQKIEWHG (SEQ ID NO:6). In some embodiments, the SCIN antigen comprises SEQ ID NO:4, SEQ ID NO: 5 and SEQ ID NO:6.

As indicated above, provided herein is a method of determining if a subject has a S. aureus infection, comprising: 1) generating a plasmablasts-secreted antibody medium by collecting peripheral blood mononuclear cells (PBMCs) from blood in a subject, removing existing antibodies from the PBMCs and culturing the antibody-free PBMCs in a medium for a period of time to allow secretion of antibodies by plasmablasts contained in the PBMCs into the medium; 2) contacting the plasmablasts-secreted antibody medium with one or more S. aureus antigens selected from the group consisting of an IsdA antigen, an IsdB antigen, a CHIPS antigen, and a SCIN antigen; 3) detecting the presence of one or more antigen-specific antibodies in the plasmablasts-secreted antibody medium at a level above a control; and 4) determining the S. aureus infection in the subject based on the detection of antigen-specific antibodies in step 3). In some embodiments, the binding of antigen-specific antibodies to one or two of the IsdA antigen, IsdB antigen, CHIPS antigen, and SCIN antigen indicates the subject has a S. aureus infection. In some embodiments, the binding of antigen-specific antibodies to three of the IsdA antigen, IsdB antigen, CHIPS antigen, and SCIN antigen indicates the subject has a S. aureus infection. In some embodiments, the binding of all of the IsdA antigen, IsdB antigen, CHIPS antigen, and SCIN antigen indicates the subject has a S. aureus infection.

Binding of antigen to an antibody may be detected using any method known to those of ordinary skill in the art. Binding of antigen to antibody may be detected using an immunoassay. Many types and formats of immunoassays are known and all are suitable for detecting the disclosed biomarkers. Examples of immunoassays are enzyme linked immunosorbent assays (ELISAs), enzyme linked immunospot assay (ELISPOT), radioimmunoassays (RIA), radioimmune precipitation assays (RIPA), immunobead capture such as assays involving distinctly colored antigen bound Lumavidin beads and phycoerythrin (PE) labelled anti-Ig, Western blotting, dot blotting, gel-shift assays, Flow cytometry, protein arrays, multiplexed bead arrays, magnetic capture, in vivo imaging, fluorescence resonance energy transfer (FRET), and fluorescence recovery/localization after photobleaching (FRAP/FLAP).

In general, immunoassays involve contacting a sample suspected of containing a molecule of interest (such as an S. aureus antigen) with an antibody to the molecule of interest or contacting an antibody to a molecule of interest (such as antibodies to an S. aureus antigen) with a molecule that can be bound by the antibody, as the case may be, under conditions effective to allow the formation of immunocomplexes. Contacting a sample with the antibody to the molecule of interest or with the molecule that can be bound by an antibody to the molecule of interest under conditions effective and for a period of time sufficient to allow the formation of immune complexes (primary immune complexes) is generally a matter of simply bringing into contact the molecule or antibody and the sample and incubating the mixture for a period of time long enough for the antibodies to form immune complexes with, i.e., to bind to, any molecules (e.g., antigens) present to which the antibodies can bind. In many forms of immunoassay, the sample-antibody composition, such as a tissue section, ELISA plate, dot blot or Western blot, can then be washed to remove any non-specifically bound antibody species, allowing only those antibodies specifically bound within the primary immune complexes to be detected.

Immunoassays can include methods for detecting or quantifying the amount of a molecule of interest (such as antibodies to an S. aureus antigen) in a sample, which methods generally involve the detection or quantitation of any immune complexes formed during the binding process. In general, the detection of immunocomplex formation is well known in the art and can be achieved through the application of numerous approaches. These methods are generally based upon the detection of a label or marker, such as any radioactive, fluorescent, biological or enzymatic tags or any other known label.

As used herein, a label can include a fluorescent dye, a member of a binding pair, such as biotin/streptavidin, a metal (e.g., gold), or an epitope tag that can specifically interact with a molecule that can be detected, such as by producing a colored substrate or fluorescence. Substances suitable for detectably labeling proteins include fluorescent dyes (also known herein as fluorochromes and fluorophores) and enzymes that react with colorimetric substrates (e.g., horseradish peroxidase). Furthermore, in the case where multiple antigens are reacted with a single array, each antigen can be labeled with a distinct fluorescent compound for simultaneous detection. Labeled spots on the array are detected using a fluorimeter, the presence of a signal indicating an antigen bound to a specific antibody.

Also provided herein is a kit for determining if a subject has a Staphylococcus aureus (S. aureus) infection, comprising one or more antigens selected from the group consisting of an IsdA antigen, an IsdB antigen, a CHIPS antigen, and a SCIN antigen; a reservoir for contacting the one or more antigens with a plasmablasts-secreted antibody medium derived from the subject; and a composition for detecting binding between the one or more antigens with one or more antibodies in the plasmablasts-secreted antibody medium. In some embodiments, one or two of the IsdA antigen, IsdB antigen, CHIPS antigen, and SCIN antigen are comprised within the kit. In other embodiments, three of the IsdA antigen, IsdB antigen, CHIPS antigen, and SCIN antigen are comprised within the kit. In other embodiments, all four of the IsdA antigen, IsdB antigen, CHIPS antigen, and SCIN antigen are comprised within the kit. The one or more antigens can be bound to a solid support in or on the reservoir in any manner. In some embodiments, the one or more antigens are bound to the reservoir itself in an array.

The one or more compositions for detecting binding between the one or more antigens with one or more antibodies in the plasmablasts-secreted antibody medium can be any known to one of ordinary skill in the art. In some embodiments, the composition for detecting binding is a labelled anti-IgG antibody. As indicated above, the word “label” when used herein includes a detectable compound or composition which can be conjugated directly or indirectly to a molecule or protein, e.g., an antibody. The label may itself be detectable (e.g., radioisotope labels or fluorescent labels) or, in the case of an enzymatic label, may catalyze chemical alteration of a substrate compound or composition which is detectable. In some embodiments, the label is fluorescent.

It should also be understood that the foregoing relates to preferred embodiments of the present invention and that numerous changes may be made therein without departing from the scope of the invention. The invention is further illustrated by the following examples, which are not to be construed in any way as imposing limitations upon the scope thereof. On the contrary, it is to be clearly understood that resort may be had to various other embodiments, modifications, and equivalents thereof, which, after reading the description herein, may suggest themselves to those skilled in the art without departing from the spirit of the present invention and/or the scope of the appended claims. All patents, patent applications, and publications referenced herein are incorporated by reference in their entirety for all purposes.

### EXAMPLES

### Example 1

### Identification of Diagnostic S. aureus Antigens

Antigen Selection.

Fourteen antigens were selected for analysis. Ten are cell-wall-associated enzymes or binding proteins and four are secreted factors that interfere with the human immune response. Additional selection criteria included: 1) conservation among S. aureus strains; 2) single subunit; 3) a role in one or more essential functions; 4) accessibility from outside the cell wall; and 5) evidence that antibody titer increased during infection. The following were several antigens that were chosen: cell wall modifying enzymes glucosaminidase (Gmd), amidase (Amd) [Gedbjerg N, LaRosa R, Hunter J G, et al. J Bone Joint Surg Am. 2013 Nov. 20; 95(22):e171; Varrone J, Dussmann E, Yi Y, et al. Orthopaedic Research Society 20122012; Varrone J, Li D, Daiss J, et al. IBMS BoneKEy 2011; 8(4):187-194; Varrone J J, Bentley K L D, Bello-Irizarry S N, et al. Journal of Orthopaedic Research. 2014; in press; Bose J L, Lehman M K, Fey P D, et al. PLoS One. 2012; 7(7):e42244; Brady R A, O'May G A, Leid J G, et al. Infect Immun. 2011 April; 79(4):1797-1803; Biswas R, Voggu L, Simon U K, et al. FEMS Microbiol Lett. 2006 June; 259(2):260-268; Gotz F, Heilmann C, Stehle T. Int J Med Microbiol. 2013 Dec. 1; Zoll S, Patzold B, Schlag M, et al. PLoS Pathog. 2010 March; 6(3):e1000807; Zoll S, Schlag M, Shkumatov A V, et al. J Bacteriol. 2012 August; 194(15):3789-3802; Buttner F M, Zoll S, Nega M, et al. J Biol Chem. 2014 Apr. 18; 289(16):11083-11094; Gotz F, Heilmann C, Stehle T. Int J Med Microbiol. 2014 March; 304(2):156-163], and sortase (SrtA) [Kim H K, Kim H Y, Schneewind O, et al. FASEB J. 2011 October; 25(10):3605-3612; Chen F, Liu B, Wang D, et al. FEMS Microbiol Lett. 2014 February; 351(1):95-103; Maresso A W, Schneewind O. Pharmacol Rev. 2008 March; 60(1):128-141], the iron-sequestering proteins IsdA [Clarke S R, Brummell K J, Horsburgh M J, et al. J Infect Dis. 2006 Apr. 15; 193(8):1098-1108; Clarke S R, Foster S J. Infect Immun. 2008 April; 76(4):1518-1526; Kim H K, DeDent A, Cheng A G, et al. Vaccine. 2010 Aug. 31; 28(38):6382-6392], IsdB [Kim H K, DeDent A, Cheng A G, et al. Vaccine. 2010 Aug. 31; 28(38):6382-6392; Torres V J, Pishchany G, Humayun M, et al. J Bacteriol. 2006 December; 188(24):8421-8429; Zapotoczna M, Jevnikar Z, Miajlovic H, et al. Cell Microbiol. 2013 June; 15(6):1026-1041] and IsdH [Foster T J, Geoghegan J A, Ganesh V K, et al. Nat Rev Microbiol. 2014 January; 12(1):49-62; Visai L, Yanagisawa N, Josefsson E, et al. Microbiology. 2009 March; 155(Pt 3):667-679], and the adhesins ClfA [Ganesh V K, Rivera J J, Smeds E, et al. PLoS Pathog. 2008 November; 4(11):e1000226; Li Y, Li Z, Li Y, et al. Wei Sheng Wu Xue Bao. 2013 Sep. 4; 53(9):966-975; Li Y, Liu Y H, Li Z J, et al. Res Vet Sci. 2013 June; 94(3):490-495; Arrecubieta C, Matsunaga I, Asai T, et al. J Infect Dis. 2008 Aug. 15; 198(4):571-575; Patti J M, Allen B L, McGavin M J, et al. Annu Rev Microbiol. 1994; 48:585-617], ClfB [Corrigan R M, Miajlovic H, Foster T J. BMC Microbiol. 2009; 9:22; Ni Eidhin D, Perkins S, Francois P, et al. Mol Microbiol. 1998 October; 30(2):245-257], FnbpA [Bingham R J, Rudino-Pinera E, Meenan N A, et al. Proc Natl Acad Sci USA. 2008 Aug. 26; 105(34):12254-12258; Edwards A M, Potts J R, Josefsson E, et al. PLoS Pathog. 2010; 6(6):e1000964; Stemberk V, Jones R P, Moroz O, et al. J Biol Chem. 2014 May 2; 289(18):12842-12851], and coagulase (Coa) [Cheng A G, DeDent A C, Schneewind O, et al. A play in four acts: Trends Microbiol. 2011 May; 19(5):225-232; Cheng A G, McAdow M, Kim H K, et al. PLoS Pathog. 2010; 6(8):e1001036].

The selected secreted antigens were alpha hemolysin (Hla) [Chua K Y, Monk I R, Lin Y H, et al. BMC Microbiol. 2014; 14:31; Bubeck Wardenburg J, Schneewind O. J Exp Med. 2008 Feb. 18; 205(2):287-294], CHIPS [Bunschoten A, Feitsma L J, Kruijtzer J A, et al. Bioorg Med Chem Lett. 2010 Jun. 1; 20(11):3338-3340; Bunschoten A, Ippel J H, Kruijtzer J A, et al. Amino Acids. 2011 February; 40(2):731-740; Postma B, Poppelier M J, van Galen J C, et al. J Immunol. 2004 Jun. 1; 172(11):6994-7001; Powers M E, Bubeck Wardenburg J. PLoS Pathog. 2014 February; 10(2):e1003871; van Wamel W J, Rooijakkers S H, Ruyken M, et al J Bacteriol. 2006 February; 188(4):1310-1315], SCIN [Jongerius I, Kohl J, Pandey M K, et al. J Exp Med. 2007 Oct. 1; 204(10):2461-2471; Jongerius I, Puister M, Wu J, et al. J Immunol. 2010 January 1; 184(1):420-425; Rooijakkers S H, Milder F J, Bardoel B W, et al. J Immunol. 2007 Sep. 1; 179(5):2989-2998; Rooijakkers S H, van Strijp J A. Mol Immunol. 2007 January; 44(1-3):23-32] and Efb [Ko Y P, Liang X, Smith C W, et al. J Biol Chem. 2011 Mar. 18; 286(11):9865-9874; Ricklin D, Ricklin-Lichtsteiner S K, Markiewski M M, et al. J Immunol. 2008 Dec. 1; 181(11):7463-7467] each of which interferes with human immune function and the signature enzyme of S. aureus.

Antigen Expression.

For each antigen the process was the same: i) a consensus sequence was created using Geneious software on S. aureus sequences compiled from GenBank; ii) sequences known or likely to be present on other S. aureus proteins were removed; iii) sequences encoding domains likely to cause problems during expression in E. coli such as those that span membranes were removed; iv) sequences encoding an N-terminal hexahistidine to facilitate purification and a C-terminal 14-amino acid AviTag biotinylation site to facilitate immobilization on Lumavidin beads were added; v) the chimeric, truncated gene was synthesized de novo using codon optimization for E. coli; vi) the synthetic gene was expressed in strains of E. coli co-expressing the biotinylating enzyme BirA [Kay B K, Thai S, Volgina V V. Methods Mol Biol. 2009; 498:185-196]; and vii) the recombinant protein was harvested and purified by metal chelation chromatography.

Human Serum Samples.

Serum samples were collected from 75 patients under a protocol approved by a Research Subjects Review Board. Forty were from non-infected control patients; 35 were from cases with culture-confirmed, deep-seated musculoskeletal infections with S. aureus. Sera were stored at −80° C. until use.

Whole blood was collected by a phlebotomist into two 10 mL green-topped sodium heparin tubes, mixed by inversion to distribute the anticoagulant, and kept between 20 and 30° C. during transit to the laboratory within four hours. 20 mL of commercially available density reagent, Polymorphprep, was added to a 50 mL conical, screw-capped tube. Then, 10-20 mL of a single patient's anti-coagulated whole blood was layered on top of the density reagent and the capped tubes were centrifuged at 500×g in a swinging bucket centrifuge kept at 25° C. for 35 minutes. At the end of the centrifugation, red blood cells were at the bottom of the tube, neutrophils were present as a band just above the red blood cells and peripheral blood mononuclear cells (PBMC), which PBMC include the circulating plasmablasts, formed a layer at the boundary of the density reagent and the plasma. The PBMC were then carefully collected by aspiration from the plasma:density agent boundary and washed four times in DMEM always keeping the temperature between 20° C. and 37° C. The washed cells were then placed in 10 mL of DMEM supplemented with fetal bovine serum (20% v/v) and gentamicin (50 μg/ml) and cultured at 37° C. for 24-72 hours in a standard cell culture incubators with 5% CO₂. Following the cell culture, the culture medium (MENSA) was harvested, centrifuged at 800×g for 5 minutes and frozen in aliquots for analysis by the multiplex immunoassay described above.

Multiplex Immunoassay.

Each purified S. aureus antigen was then immobilized on a distinctly colored preparation of Lumavidin beads to yield a preparation of beads that displays only one antigen and possesses a unique color signature in flow cytometry. The antigen-laden Lumavidin beads were then combined and incubated with MENSA, washed, incubated with a secondary reagent: phycoerythrin(PE)-conjugated goat anti-human IgG (or IgM), and then analyzed on a Bio-Plex 200 (BioRad). The Fluorescence Intensity (FI) of PE associated with each color bead was measured as the antigen specific signal. The mean of 50-100 beads of each specificity is reported herein. Titers are expressed as multiples of the median of the control population; positive control is 10,000-fold diluted pool of sera reactive with all 14 antigens.

Antibodies in Serum are Poor Predictors of Infection.

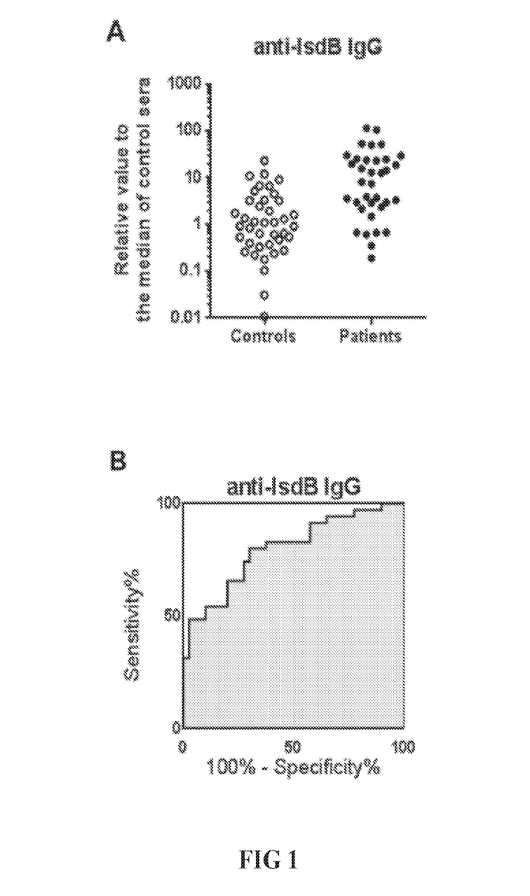

Representative data from the best single antigen predictor of recent or ongoing infection, the iron-sequestering cell wall protein IsdB, is presented in FIG. 1. Several features are immediately apparent. First, the control population has measurable levels of anti-IsdB and the range of titers exceeds two orders of magnitude. Second, the infected patients have a similarly wide distribution of values that is about a half an order of magnitude higher. Thus there is a high, noisy background level in the non-infected controls and a small, noisy elevation in titers of the infected patients. The dot plot in FIG. 1A presents this clearly and the derived ROC curve in FIG. 1B illustrates that the clinical predictive power is modest (Area-under-the-curve (AUC)=0.80)). Combinations of antibody titers improve clinical prediction slightly: multivariate analysis of titers for all 14 antigens yield an AUC just under 0.90. Titers for IgM had no predictive power, AUC 0.5-0.55. (data not shown).

Initial Data Using Circulating Plasmablasts is Encouraging: 100% Sensitivity, 100% Specificity in a Small Cohort.

The potential utility, simplicity and economy of using the host's immune response as a clinical predictor of ongoing infection is lost in the jaws of high, noisy background and low, noisy signal. The hypothesis set forth herein is that one can break through this constraint by measuring the antibody secreted in vitro by circulating plasmablasts which are present only during active infection. To test this idea, whole blood was collected from patients with culture-confirmed prosthetic joint infections and from patients presenting in the Emergency Department who were subsequently admitted to Orthopaedics with suspected implant-associated infections. The peripheral blood mononuclear cells (PBMCs) which contain circulating plasmablasts were harvested and placed in culture for 72 hours as described above. Then the culture medium enriched for newly synthesized antibody (MENSA) was obtained and the abundance of IgG for each of the 14 antigens was measured in the multiplex immunoassay described above.

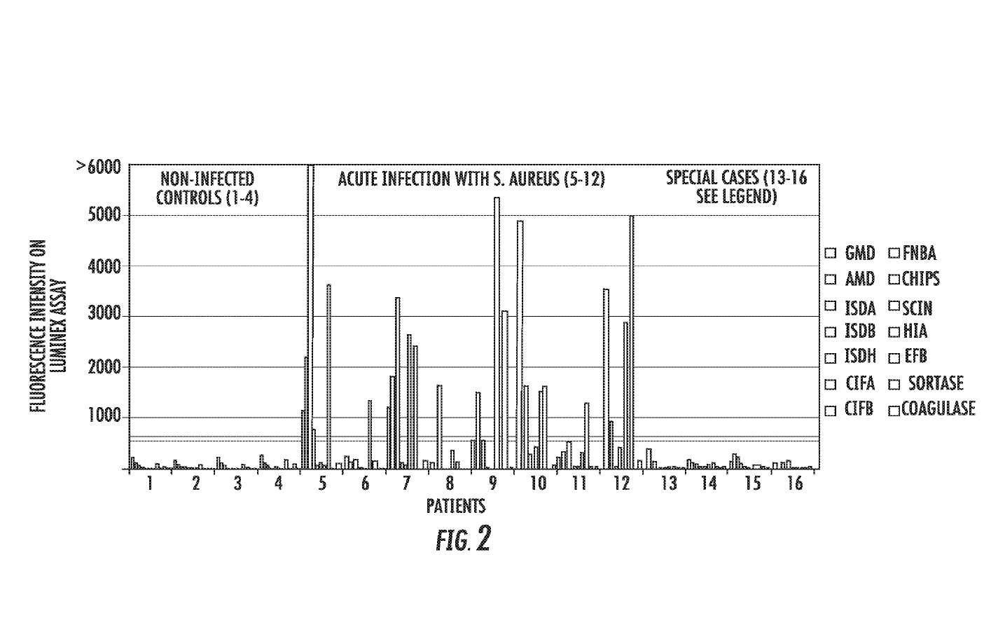

The results from the first sixteen patients are presented in FIG. 2. The Fluorescence Intensity (FI) values obtained from undiluted MENSA for each of the 14 antigens are presented as colored vertical bars. Patients numbered 1-4 were non-infected controls and one can see that they have very little IgG against any of the antigens. The origin of the tiny signals observed is under investigation and may be completely eliminated. Patients numbered 5-12 each had ongoing culture-confirmed infections and each had MENSA bearing IgG for one or more of the 14 antigens. Patients numbered 13-16 illustrate other conditions that should be negative. Patient number 13 had a culture-confirmed P. aeruginosa infection; Patient number 16 remained culture negative for any pathogen. Patients numbered 14 and 15 had culture-confirmed S. aureus infections several months before and had been on extensive antibiotic therapy for at least 30 days prior to our sampling, a promising result suggesting the ASC decline when therapy is successful.

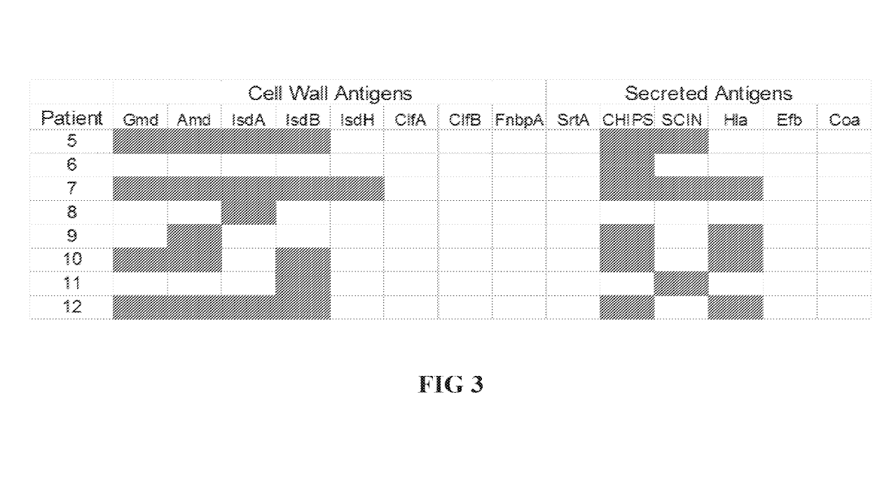

As shown in FIG. 3, only three antigens were required to identify all eight of the infected patients (CHIPS, IsdA and (IsdB or SCIN)). The response to each antigen in FIG. 2 was considered positive if it exceeded the highest value for any antigen among the controls by at least a factor of two (shown as red squares in FIG. 3) and negative if it did not (shown as white squares in FIG. 3).

SEQUENCE LISTING

SEQ ID NO:1
GATEATNATNNQSTQVSQATSQPINFQVQKDGSSEKSHMDDYMQHPGKVI
KQNNKYYFQTVLNNASFWKEYKFYNANNQELATTVVNDNKKADTRTINVA
VEPGYKSLTTKVHIVVPQINYNHRYTTHLEFEKAIPTLADAAKPNNVKPV
QPKPAQPKTPTEQTKPVQPKVEKVKPTVTTTSKVEDNHSTKVVSTDTTKD
QTKTQTAHTVKTAQTAQEQNKVQTPVKDVATAKSESNNQAVSDNKSQQTN
KVTKHNETPKQAGPSKDSKAKELPKTG
SEQ ID NO: 2
MSNGEAQAAAEETGGTNTEAQPKTEAVASPTTTSEKAPETKPVANAVSVS
NKEVEAPTSETKEAKEVKEVKAPKETKEVKPAAKATNNTYPILNQELREA
IKNPAIKDKDHSAPNSRPIDFEMKKKDGTQQFYHYASSVKPARVIFTDSK
PEIELGLQSGQFWRKFEVYEGDKKLPIKLVSYDTVKDYAYIRFSVSNGTK
AVKIVSSTHFNNKEEKYDYTLMEFAQPIYNSADKFKTEEDYKAEKLLAPY
KKAKTLERQVYELNKIQDKLPEKLKAEYKKKLEDTKKALDEQVKSAITEF
QNVQPTNEKMTDLQDTKYVVYESVENNESMMDTFVKHPIKTGMLNGKKYM
VMETTNDDYWKDFMVEGQRVRTISKDAKNNTRTIIFPYVEGKTLYDAIVK
VHVKTIDYDGQYHVRIVDKEAFTKANTDKSNKKEQQDNSAKKEATPATPS
KPTPSPVEKESQKQDSQKDDNKQLPSVEKENDASSESGKDKTPATKPTKG
EVESSSTTPTKVVSTTQNVAKPTTASSKTTKDVVQTSAGSSEAKDSAPLQ
KANIKNTNDGHTQSQNNKNT
SEQ ID NO: 3
FTFEPFPTNEEIESNKKMLEKEKAYKESFKNSGLPTTLGKLDERLRNYLK
KGTKNSAQFEKMVILTENKGYYTVYLNTPLAEDRKNVELLGKMYKTYFFK
KGESKSSYVINGPGKTNEYAY
SEQ ID NO: 4
STSLPTSNEYQNEKLANELKSLLDELNVNELATGSLNTYYKRTIKISGQK
AMYALKSKDFKKMSEAKYQLQKIYNEIDEALKSY
SEQ ID NO: 5
MHHHHHH
SEQ ID NO: 6
LNDIFEAQKIEWHG

1. A method of determining an S. aureus infection in a subject, comprising:
a. generating a plasmablasts-secreted antibody medium by collecting peripheral blood mononuclear cells (PBMCs) from blood in a subject, removing existing antibodies from the PBMCs and culturing the antibody-free PBMCs in a medium for a period of time to allow secretion of antibodies by plasmablasts contained in the PBMCs into the medium;
b. contacting the plasmablasts-secreted antibody medium with one or more S. aureus antigens;
c. detecting the presence of one or more antigen-specific antibodies in the plasmablasts-secreted antibody medium at a level above a control; and
d. determining the S. aureus infection in the subject based on the detection of the antigen-specific antibodies in step c.

2. The method of claim 1, wherein the one or more S. aureus antigens are selected from an IsdA antigen, an IsdB antigen, a CHIPS antigen, and a SCIN antigen.

3. The method of claim 1, wherein the plasmablasts-secreted antibody medium is contacted with one, or two, or three, or all of the IsdA antigen, IsdB antigen, CHIPS antigen, and SCIN antigen.

4. The method of claim 3, wherein the plasmablasts-secreted antibody medium is contacted with all of the IsdA antigen, IsdB antigen, CHIPS antigen, and SCIN antigen.

5. The method of claim 3, wherein the plasmablasts-secreted antibody medium is contacted with three of the IsdA antigen, IsdB antigen, CHIPS antigen, and SCIN antigen.

6. The method of claim 3, wherein the plasmablasts-secreted antibody medium is contacted with two of the IsdA antigen, IsdB antigen, CHIPS antigen, and SCIN antigen.

7. The method of claim 1, wherein the antibody-free PBMCs are cultured for about 36 to about 72 hours.

8. The method of claim 7, wherein the antibody-free PBMCs are cultured for about 72 hours.

9. The method of claim 1, wherein the presence of the one or more antigen-specific antibodies in the plasmablasts-secreted antibody medium is at a level of at least about 10% more than the control level.

10. A kit for determining if a subject has a Staphylococcus aureus (S. aureus) infection, comprising:
a. one or more antigens selected from the group consisting of an IsdA antigen, an IsdB antigen, a CHIPS antigen, and a SCIN antigen;
b. a reservoir for contacting the one or more antigens with the plasmablasts-secreted antibody medium of claim 1; and
c. a composition for detecting binding between the one or more antigens with the one or more antibodies in the plasmablasts-secreted antibody medium.

11. The kit of claim 10, wherein the one or more antigens are all of the IsdA antigen, IsdB antigen, CHIPS antigen, and SCIN antigen.

12. The kit of claim 10, wherein the one or more antigens are bound to the reservoir in an array.

13. The kit of claim 10, wherein the composition for detecting binding comprises one or more labelled anti-IgG antibodies.

14. The kit of claim 13, wherein the label is fluorescent.
